(19) United States
(12) Patent Application Publication
(10) Pub. No.: US 2026/0013891 A1
(43) Pub. Date: Jan. 15, 2026

(54) MEDICAL INSTRUMENT HANDLE WITH QUICK ASSEMBLY AND OVERLOAD PROTECTION FEATURES

(71) Applicant: Unintech GmbH, Düsseldorf (DE)

(72) Inventor: QINGUANG YOU, Düsseldorf (DE)

(73) Assignee: Unintech GmbH, Düsseldorf (DE)

(21) Appl. No.: 19/074,888

(22) Filed: Mar. 10, 2025

(30) Foreign Application Priority Data
Jul. 11, 2024 (DE) DE202024103865.7

(51) Int. Cl. A61B 17/29; A61B 17/00; A61B 17/295

(52) U.S. Cl. CPC A61B 17/2909; A61B 17/295; A61B 2017/00469; A61B 2017/00477; A61B 2017/2925; A61B 2017/2946

(57) ABSTRACT

The present invention relates to a handle with an overload protection feature enabling rapid assembly and disassembly. During cutting operation, overload protection is provided through a handle structure having movable and fixed handle connected together, along with a hollow outer tube radially fixed at rear end to front end of fixed handle. An internal connecting rod is axially inserted into both outer tube and fixed handle. The handle can be rapidly assembled and disassembled, including a handle and a trigger mounted on the handle. The upper end of handle has a quick-mounting interface. Connecting rod is mounted to slide within connection rod cavity, and trigger's actuation end penetrates the connection rod cavity, attaching to the rear end of the connecting rod. This handle can be combined with various types of medical forceps heads to perform different functions and enables quick assembly and disassembly, easy handling, and precise operation.

### FIELD

This invention relates to medical instrument handles, particularly to a handle device designed for overload protection and rapid assembly/disassembly of medical instruments. This handle can be paired with different instrument heads to perform various functions, offering ease of use, precise action, and quick operational readiness.

### BACKGROUND

Medical instruments like scrapers, chisels, and bone punches are often hand-operated with handles requiring quick attachment or detachment during surgical procedures. The speed and convenience of switching between instruments can significantly impact surgical outcomes. Existing surgical instruments typically rely on screws for handle attachment, which can be time-consuming and inconvenient, especially during emergency procedures. For example, Chinese patent CN202311721606X discloses a laparoscopic instrument with a non-detachable head, which limits swift instrument switching.

### SUMMARY

The present invention relates to the field of medical instruments and introduces a handle with an overload protection feature that enables rapid assembly and disassembly. During the cutting operation, it provides overload protection through a handle structure that comprises a movable and a fixed handle connected together, along with a hollow outer tube radially fixed at the rear end to the front end of the fixed handle. An internal connecting rod is axially inserted into both the outer tube and the fixed handle. The front end of the push rod is connected to the movable working component of the cutting instrument, and the rear end of the push rod is axially introduced into the connecting rod, where both parts establish an interlocking connection. This design improves tension control during bone and tissue cutting, enhances cutting efficiency, and ensures safety in minimally invasive procedures without requiring modifications to the structure or inner diameter of existing endoscopic instrument channels.

The assembly process allows for rapid assembly and disassembly, including a handle and a trigger mounted on the handle. The upper end of the handle has a quick-mounting interface, consisting of a connection rod cavity fixed at the top of the handle and a connecting sleeve mounted at the front end of the connection rod cavity. Inside the connecting sleeve, an eccentric locking ring is positioned. The connecting rod is mounted to slide within the connection rod cavity, and the trigger's actuation end penetrates the connection rod cavity, attaching to the rear end of the connecting rod. The front end of the connecting rod features a self-locking interface designed to attach a drive rod for medical instruments. This handle can be combined with various types of medical forceps heads to perform different functions and enables quick assembly and disassembly, easy handling, and precise operation.

### BRIEF DESCRIPTION OF THE DRAWINGS

The accompanying drawings illustrate one or more embodiments of the present disclosure, and features and benefits thereof, and together with the written description, serve to explain the principles of the present invention. Wherever possible, the same reference numbers are used throughout the drawings to refer to the same or like elements of an embodiment, and wherein:

FIG. 1 is a schematic view of a medical instrument handle in its default position according to certain embodiments of the present disclosure;

FIG. 2 Shows an enlarged view of a connection rod cavity and a connecting sleeve according to certain embodiments of the present disclosure;

FIG. 3 illustrates an exploded view of a quick-mounting interface and a self-locking mechanism according to certain embodiments of the present disclosure;

FIG. 4 shows the handle during the assembly of a medical instrument head according to certain embodiments of the present disclosure; and

FIG. 5 demonstrates the medical instrument handle in use with the trigger engaged according to certain embodiments of the present disclosure.

### DETAILED DESCRIPTION

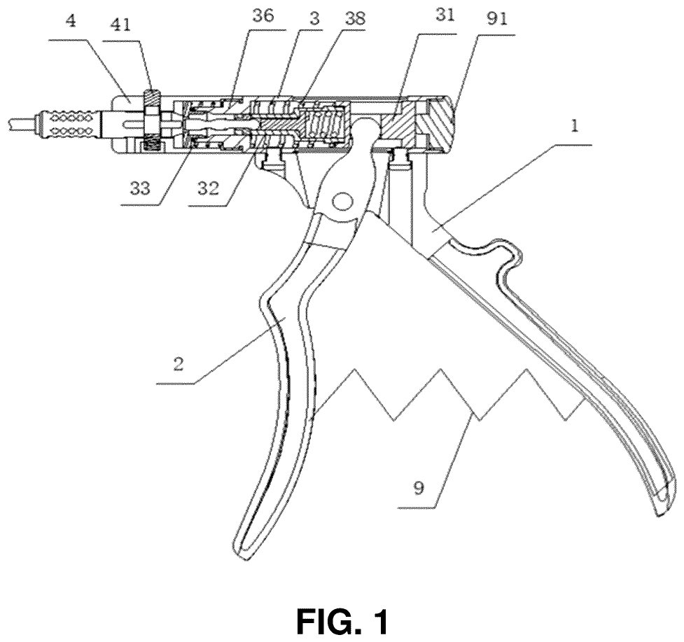

Referring to FIG. 1, the quick-assembly medical instrument handle comprises a handle (1) with an attached trigger (2) for easy assembly of medical instrument heads. The handle's upper end features a quick-mounting interface that includes a connection rod cavity (3) and a connecting sleeve (4). These elements facilitate secure and efficient attachment of various instrument heads.

The connection sleeve (4) houses an eccentric locking ring (41) that secures the outer sleeve of the instrument head in place during use. The eccentric locking ring (41) is actuated by a button (43) on the connecting sleeve (4), allowing the locking ring to move within an annular groove and secure or release the instrument head as needed.

The connecting rod (31) slides within the connection rod cavity (3), connecting to the rear end of the instrument's drive rod. The self-locking interface ensures a stable connection, and an eccentric locking ring (41) on the connecting sleeve secures various instrument heads.

The technical solution of this invention will be described in detail below with reference to the accompanying drawings, illustrating one preferred embodiment of the medical instrument quick-assembly handle.

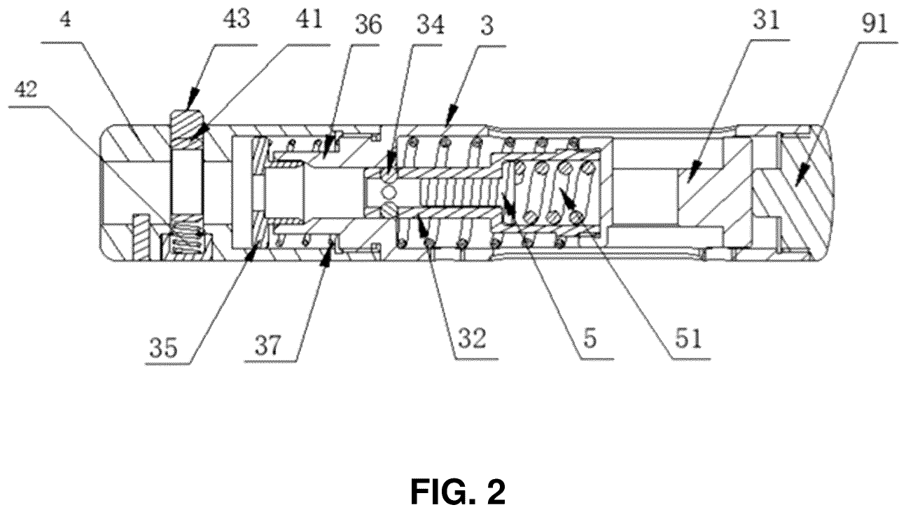

Referring to FIG. 1 and FIG. 2, the quick-mounting interface is composed of a connection rod cavity (3) secured at the upper end of handle (1) and a connecting sleeve (4) installed at the front end of the connection rod cavity (3). These components are coaxially aligned and can be joined via snap fit, threading, or ultrasonic welding, as depicted. The rear end of the connection rod cavity (3) is sealed with a screw cap (91), facilitating easy disassembly and cleaning of internal parts. Within the connecting sleeve (4), an eccentric locking ring (41) is fitted to grip the outer sleeve of the medical instrument head, providing firm fixation.

The connecting rod (31) slides within the connection rod cavity (3) and connects to the trigger (2). The exterior wall of the connection rod cavity (3) includes several elongated holes (39), one of which accommodates the trigger (2), allowing the trigger to move along the cavity's axis. These elongated holes also allow for internal cleaning.

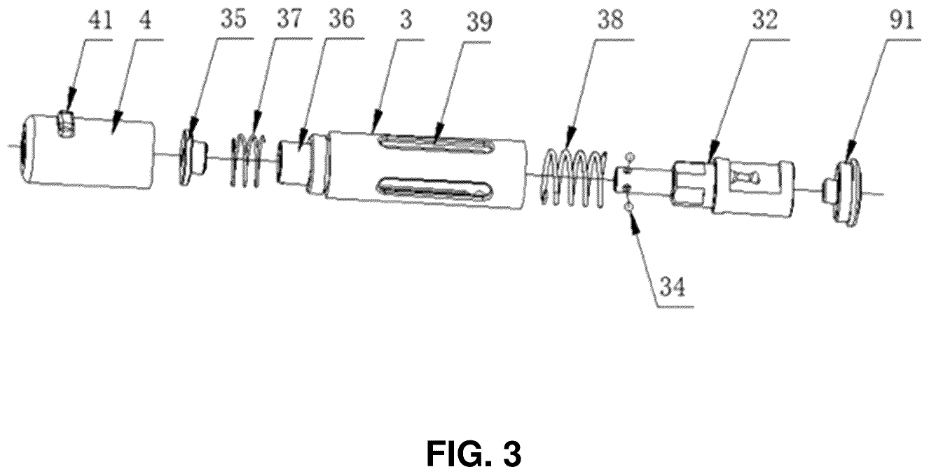

The self-locking interface, as shown in FIG. 2 and FIG. 3, comprises a connection tube (32) mounted at the front end of the connecting rod (31), a compression ring (33) within the connecting sleeve (4), and a set of balls (34) distributed around the connection tube's outer wall. The compression ring (33) is configured with a spring holder (35) at one end and a tapered opening at the other. The balls (34) are positioned within mounting holes on the connection tube (32) such that they project slightly into the tube interior, providing a clamping effect.

The core tube (36), as shown in FIG. 3, extends from the front end of the connection rod cavity (3) into the connecting sleeve (4). This core tube has a stepped shape, with a larger inner diameter at its front end, allowing for easy insertion of the connection tube (32) and the compression ring (33). When no external force is applied, a compression spring (37) between the spring holder (35) and the core tube (36) pushes the compression ring away from the core tube, holding the balls in position to engage the drive rod's end.

### Operation and Assembly Process

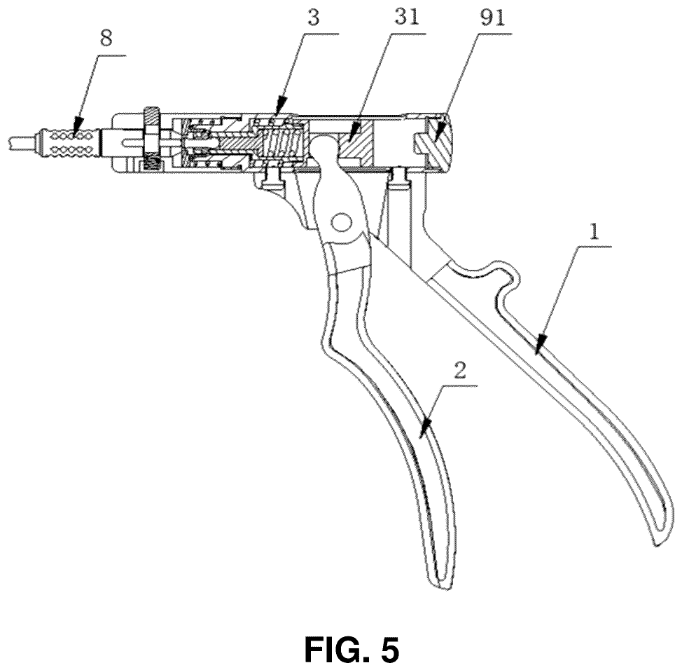

As shown in FIG. 5, in certain embodiments, in operation, the handle (1) is held, and the trigger (2) is actuated to move the connecting rod (31) and connection tube (32) forward, compressing the spring (38) that surrounds the connection tube (32) and returning the connecting rod and connection tube to their original positions when released. When a medical instrument head is being assembled, the eccentric locking ring (41) secures the head within the connecting sleeve (4) and engages with the outer sleeve of the instrument head, ensuring a firm fit.

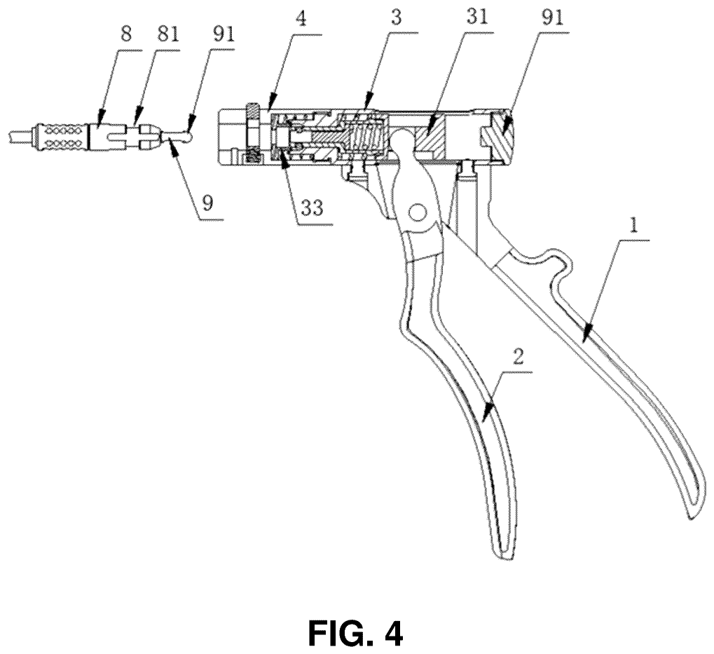

The buffer plug (5), located inside the connection tube (32) and supported by a buffer spring (51), provides axial limitation and shock absorption during assembly. As shown in FIG. 4, when the instrument head is fully engaged, the eccentric locking ring (41) maintains the head's position, while the compression ring (33) and buffer plug (5) offer additional stability and dampening.

After the trigger (2) is released, the components are reset to their original positions, allowing the balls (34) to roll along the core tube's inner wall, reducing friction and facilitating smooth disassembly when the medical instrument head is removed.

### Advantages of the Invention

The invention provides numerous benefits for medical procedures. The handle's compatibility with various instrument heads increases versatility and efficiency, allowing for quick changes between different surgical functions. This design reduces operation time and minimizes patient discomfort. Additionally, the structure enhances precise control and reduces the risk of procedural complications.

The overload protection feature prevents tool breakage, reducing surgical risks. The adjustable bite force can be regulated for various materials, improving the tool's adaptability and functionality. Moreover, the handle's components are easily disassembled for cleaning and maintenance, supporting strict hygiene standards.

This handle design enhances the surgical workflow by enabling surgeons to switch tools efficiently, leveraging their skills more effectively, and increasing the likelihood of successful outcomes.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope. Accordingly, the scope of the present disclosure is defined by the appended claims rather than the foregoing description and the exemplary embodiments described therein.

What is claimed is:

1. A medical instrument quick-assembly handle, Comprising:
a handle and a trigger mounted on the handle, wherein an upper end of the handle includes a quick-mounting interface, having a connection rod cavity fixed at a top of the handle and a connecting sleeve mounted at a front end of the connection rod cavity, an eccentric locking ring is positioned inside the connecting sleeve, a connecting rod is slidingly mounted within the connection rod cavity, and an actuation end of the trigger extends through the connection rod cavity and connects to a rear end of the connecting rod, where the front end of the connecting rod comprises a self-locking interface for attaching a drive rod of medical instruments.

2. The medical instrument quick-assembly handle according to claim 1, wherein the self-locking interface comprises:
a connection tube affixed to the front end of the connecting rod;
a compression ring slidingly mounted inside the connecting sleeve, wherein the compression ring includes a spring holder; and
a plurality of balls mounted on an outer wall of the connection tube,
wherein the front end of the connection rod cavity includes a core tube projecting into the connecting sleeve, a front inner diameter is larger than its rear end, allowing the connection tube to be inserted into the core tube, and a compression spring is positioned between the spring holder and the core tube.

3. The medical instrument quick-assembly handle according to claim 1, wherein the actuation end of the trigger comprises a ball head, and the connecting rod comprises a corresponding ball socket to accommodate the ball head.

4. The medical instrument quick-assembly handle according to claim 1 further comprising a connection tube spring, wherein the connection tube spring surrounds the connection tube and is positioned between the core tube and the connection tube.

5. The medical instrument quick-assembly handle according to claim 1, wherein the eccentric locking ring comprises an annular groove configured to accommodate the eccentric locking ring, a first side of the eccentric locking ring comprises a button that protrudes beyond the outer wall of the connecting sleeve, and an opposite side of the eccentric locking ring houses a retaining spring in the annular groove to bias the eccentric locking ring toward the button.

6. The medical instrument quick-assembly handle according to claim 1, wherein a buffer plug is positioned in an internal space of the connection tube between the connecting rod and a buffer spring.
